(19) United States
(12) Patent Application Publication
(10) Pub. No.: US 2026/0207792 A1
(43) Pub. Date: Jul. 23, 2026

(54) CONTRAST AGENT FOR MAGNETIC RESONANCE IMAGING

(71) Applicant: WENZHOU INSTITUTE, UNIVERSITY OF CHINESE ACADEMY OF SCIENCES, Zhejiang (CN)

(72) Inventors: Fangfu YE, Zhejiang (CN); Haiping FANG, Zhejiang (CN); Xin ZHANG, Zhejiang (CN)

(21) Appl. No.: 19/144,364

(22) PCT Filed: Dec. 29, 2022
(86) PCT No.: PCT/CN2022/143617

§ 371 Date: Jun. 27, 2025

(51) Int. Cl. A61K 49/12; A61K 49/18

(52) U.S. Cl. CPC A61K 49/126; A61K 49/1803

(57) ABSTRACT

A contrast agent for magnetic resonance imaging (MRI) is discloses. The MRI contrast agent includes a paramagnetic polymer prepared by mixing the sp²-hybridized matrix and the salt solution of Ca cations. The MRI contrast agent is not only biocompatible and nontoxic, but also displays equivalent or better contrast effect compared with conventional commercial contrast agents.

### CROSS-REFERENCE TO RELATED APPLICATIONS

This application is the U.S. National Stage entry of International Application No. PCT/CN2022/143617, filed on Dec. 29, 2022, which is hereby incorporated herein by reference in its entirety for all purposes.

### TECHNICAL FIELD

The invention relates to a contrast agent for magnetic resonance imaging (MRI).

### BACKGROUND OF THE INVENTION

Magnetic resonance imaging (MRI) is a commonly used non-invasive medical imaging method, which has many advantages such as no radiation and high resolution. It plays an important role in the diagnosis and prognosis of a broad range of diseases. By setting a magnetic field of specific intensity, MRI uses radio-frequency pulses to trigger nuclear resonance of hydrogen atoms in the body and hydrogen atoms will absorb energy. When the radio-frequency pulses stop, the hydrogen nuclei will emit radio signals at specific frequencies and release the absorbed energy. These signals can be processed electronically to create a simulated image of the inside of the human body. The process of the nucleus recovering from the excited state to the ground state is called the relaxation process. Relaxation time, a certain amount of time required by the relaxation process, reflects the interaction between protons and their surroundings in the proton system. The relaxation time is an inherent characteristic of the organism.

Clinical studies have found that the relaxation time of some different healthy tissues or tumor tissues overlap with each other, resulting in poor imaging and thus inaccurate diagnosis. Therefore, exogenous contrast agents are applied to change the relaxation characteristics of local tissues, and then enhance image contrast and improve image resolution, so as to achieve good clinical imaging effect (J. Wahsner et al. 2019). At present, MRI contrast agents have wide application. According to statistics, more than 40 million enhanced magnetic resonance scans are performed worldwide each year (V. M. Runge 2017).

Generally, depending on the operational mode, MRI contrast agents can be categorized as T1-weighted contrast agents and T2-weighted contrast agents. T1-weighted contrast agents, also called positive contrast agents, increase the signal intensity on T1-weighted images and brighten the accumulation area by shortening longitudinal relaxation times. T2-weighted contrast agents, also called negative contrast agents, weaken the signal intensity and darken the immediate and surrounding area by shortening transversal relaxation times. Currently, the most commonly used contrast agents in clinics are T1-weighted contrast agents. According to the magnetic composition, MRI contrast agents can be categorized as paramagnetic contrast agents, superparamagnetic contrast agents and ferromagnetic contrast agents. Commonly used MRI contrast agents are paramagnetic positive contrast agents, such as gadolinium-based contrast agents and manganese-based contrast agents.

Conventional commercial paramagnetic MRI contrast agents are composed of paramagnetic metal ions and ligands. Paramagnetic metal ions are chelated to ligands to increase in vivo metabolic rate of the metal ion (such as Gd³⁺ or Mn²⁺) and reduce associated toxicity of the free metal ion. However, a large number of clinical cases showed that these paramagnetic metallic complexes are not free of toxicity problems. For example, excess manganese form deposits in brain areas, such as the substantia nigra and basal ganglia, leading to brain cell death and other symptoms such as parkinsonism. Gadolinium chelates may induce anaphylactic reaction, leading to nausea, edema, and even shock. Besides, they may cause varying degrees of damage to renal or kidney function, and in severe cases cause irreversible nephrogenic systemic fibrosis (NSF). On the other hand, these metal elements accumulate in urban drainage systems and may affect ecosystems when heavily used and discarded. Therefore, to reduce the toxicity and adverse environmental impact of commercial contrast agents, clinically standard dose for commercial contrast agents, such as: Gd-DTPA (trade name Magnevist), is usually set at 0.1 mmol/kg. In recent years, MRI contrast agent research has been focusing on the development of new ligands. For example, CN105497921A discloses a Mn²⁺-containing composition of mannitol, meglumine, polyvinylpyrrolidone. This composition can be used as an MRI contrast agent. The Mn²⁺-containing contrast agent was applied to the rat liver, and the MR imaging showed that T1-weighted images were significantly brighter, and the signal intensity is over 2 times higher than the blank signal. CN112826945B discloses a nanoscale coordination polymer for use as an MRI contrast agent. This nanoscale coordination polymer, Hemin@Gd-NCPs, has better contrast enhancement compared with Magnevist, a clinically used commercial MRI contrast agent. In addition, CN109513014B provides an MRI contrast agent with good safety profile and long circulation time in vivo. This MRI contrast agent is composed of tubulin and gadolinium elements. The gadolinium ions, when binding to tubulin, play a major role in imaging. The strong binding to tubulin avoids gadolinium ion leakage, thereby improving the biocompatibility of contrast agents. Although the three above-mentioned novel MRI contrast agents enhance the imaging capability or improve the biocompatibility, toxic gadolinium cations or manganese cations are still used.

Therefore, with the wide use of MRI in medicine, there is an urgent need for a novel class of contrast agents that can enhance image contrast and is nontoxic to humans, to help physicians complete the diagnosis and perform the operation efficiently and precisely.

### SUMMARY OF THE INVENTION

It is an object of the present invention to provide an MRI contrast agent that is not only biocompatible, but also has the equivalent or better contrast effect compared with conventional commercial contrast agents. More specifically, it is an object of the present invention to provide an MRI contrast agent comprising a paramagnetic polymer.

The theoretical computation of the paramagnetic polymer of the present invention is based on spin-polarized density function theory (DFT) calculations: when the calcium cation substitutes the cation in the matrix material, the Ca cation forms the bond in an identical length of 2.26 Å with the sp²-hybridized O atom in the carbon-oxygen double bond (—C═O). Further analyzing the bound based on electron localization function (ELF), there is a bell-shaped localization on the O atom (FIG. 1A), indicating that the bond is in the ionic state and the electron transfers from Ca to O. Employing the spin-polarized DFT calculation of molecular orbitals, it is found that the spin up HOMO (highest occupied molecular orbital) and LUMO (lowest unoccupied molecular orbital) are fully contributed by the Ca cation; whereas, the spin down HOMO and LUMO are contributed by the Ca cation and the matrix molecule containing —C—O, respectively (FIG. 1B). The asymmetry between the spin up and spin down leads to a 1.0 μB magnetic momentum on the Ca cation, resulting in the polymer product with strong paramagnetism.

In the present invention, matrix containing sp²-hybridized groups, e.g., those with carbon-oxygen double bonds, is used to adsorb Ca cations to form strongly paramagnetic polymers. The size of the sp²-hybridized matrix polymer can be such easily controlled that there is enough space for the formation of large magnetic domains in the polymer, thereby the magnetic response of the polymer per unit volume is increased in an exhaustive manner.

Unexpectedly, the above polymer product exhibits strong paramagnetism at room temperature. These materials are not consistent with the common understanding of magnetic materials in the art.

Based on the above principles, it is an object of the present invention to provide an MRI contrast agent, which comprises a paramagnetic polymer prepared by mixing the sp²-hybridized matrix and the salt solution of Ca cations.

In some embodiments, the solid content of the paramagnetic polymer ranges between 0.2-2 mg/ml.

In some embodiments, the sp²-hybridized matrix comprises carbon-oxygen double bonds (—C═O).

In some embodiments, the sp²-hybridized matrix comprises at least one sp²-hybridized group selected from the group consisting of carboxyl group, aldehyde group or acyl group.

In some embodiments, the sp²-hybridized matrix is selected from one or more of polysaccharide uronic acid, acyl and ketone.

In some embodiments, the sp²-hybridized matrix is selected from one or more of alginate, carboxymethyl chitosan, polyacrylamide, N-isopropyl acrylamide and hyaluronate.

In some embodiments, the sp²-hybridized matrix is sodium alginate.

In some embodiments, the solution concentration of the sp²-hybridized matrix ranges from 0.3 wt ‰ to 1.3 wt ‰, preferably from 0.3 wt ‰ to 0.7 wt ‰. The concentration of the Ca cation solution ranges from 0.3M to 7M, preferably from 0.3M to 6M.

In some embodiments, a microfluidic method is used for preparation of the paramagnetic polymer wherein one of the sp²-hybridized matrix and the Ca cation solution is dropped into the other at a constant rate.

In some embodiments, a mechanical stirring method is used for preparation of the paramagnetic polymer wherein the sp²-hybridized matrix is mixed with the Ca cation solution by mechanical stirring.

The paramagnetic polymer of the present invention is prepared by mixing the 0.3 wt ‰ 0-1.3 wt ‰ sp²-hybridized matrix material with the 0.3M-7M Ca cation solution. In this way, the paramagnetic polymer of the present invention exhibits strong paramagnetism. When the concentration of the sp²-hybridized matrix solution exceeds the above range, the polymer product is too stiff due to high reaction rate. When the concentration of the sp²-hybridized matrix solution is below the above range, the polymer product is hardly observed by naked eyes. If the concentration of Ca cation solution is below the above range, the polymer with paramagnetism doesn't form.

Mechanical stirring method or microfluidic method can be used for the purpose of the preparation of the present invention. By the use of these two methods, the size of the polymer product can be controlled, and the sp²-hybridized matrix can be thoroughly mixed with the Ca cation solution. Therefore sp²-hybridized groups fully mixed with Ca cations, distribute evenly in the polymer, thus generating strong paramagnetism.

The paramagnetic polymer of the present invention can be purified by the following steps. Firstly, ultrasonically disperse the suspension of the paramagnetic polymer for more than 10 minutes after the preparation of the present invention. Then place the suspension container next to a cuboid neodymium magnet (64 mm×54 mm×36 mm), which has a maximum surface magnetic field of ~0.5 T. The paramagnetic polymers will be attracted towards the magnet and accumulate on the wall of the container. Next use a micropipette to extract these accumulated paramagnetic polymers, and centrifuge for 3 min at 3000 rpm. Aspirate the supernatant, and dilute polymers for ~10 times by volume via adding the Millipore water. Repeat the washing procedure for 3 times to get the purified paramagnetic polymers.

The paramagnetic polymer used as a contrast agent provided by the present invention is biocompatible and nontoxic, and has equivalent or better contrast effects compared with conventional commercial contrast agents. When used as an MRI contrast agent, the paramagnetic polymer can be suspended in physiological saline, and the range of the solid component in polymer in the suspension is 0.2-2 mg/ml.

According to clinical needs, the diffusivity of the paramagnetic polymer of the present invention can be altered by adjusting its size, to control the contrast range and the action time of the contrast agent. For example, paramagnetic polymers can be broken up by high-speed shearing to produce polymers with a small size (~50 μm). Smaller paramagnetic polymer size leads to a higher diffusivity and makes it easier to be absorbed by the body, but the action time of the contrast agent is shortened. On the other hand, larger paramagnetic polymer size makes it easier to adjust the imaging range, and leads to a longer action time. Therefore, compared with commercial contrast agents, the paramagnetic polymer used as an MRI contrast agent of the present invention has the advantages of controllable in-situ contrast and clearer and brighter local contrast.

### BRIEF DESCRIPTION OF THE DRAWINGS

FIG. 1 depicts theoretical computations of Alg-Ca hydrogel, in which (A) shows electron localization function (ELF) calculation for the Alg-Ca system with an iso-surface of 0.4 atomic unit, which shows the bell-shaped localization on the O atom, as labelled in the red dashed rectangle; (B) shows the spin up (left column) and spin down (right column) HOMO (highest occupied molecular orbital) and LUMO (lowest unoccupied molecular orbital) of the Alg-Ca system with an iso-surface of 0.005 atomic unit.

FIG. 2 depicts the mass magnetic susceptibilities of matrices with and without carbon-oxygen double bonds (—C═O) in CaCl₂) solution and water.

FIG. 3 depicts the relaxation rate of commonly used commercial contrast agents in 1.5T magnetic field in water at 37° C.

FIG. 4 (A) depicts the T₁ relaxation time of Alg-Ca hydrogels of the present invention and commercial contrast agents in water; FIG. 4 (B) depicts the efficiency of shortening T₁ relaxation time of water by Alg-Ca hydrogels of the present invention and commercial contrast agents.

FIG. 5 depicts T₁ relaxation times and MRI phantom images of Alg-Ca hydrogels of the present invention and the commercial contrast agent Gd-DTPA in 114 μT ultralow magnetic field.

FIG. 6 depicts T₁ relaxation times and MRI phantom images of Alg-Ca hydrogels of the present invention in different liquids.

FIG. 7 depicts MR images of left and right back limbs of Sprague-Dawley rat (under anesthesia) before and after injection of Alg-Ca paramagnetic hydrogels.

FIG. 8 depicts in-situ MR images of a Huh7 tumor in the rat before and after injection of Alg-Ca paramagnetic hydrogels.

FIG. 9 depicts the cell viability of NIH-3T3 cells cultured with cell culture medium containing different volumetric concentrations of Alg-Ca hydrogels of the present invention.

FIG. 10 depicts the in vivo biocompatibility study of Alg-Ca paramagnetic hydrogels of the present invention, in which (A) shows the schematic illustration of the mouse models for safety evaluation; (B) shows the increased body weight of mice; (C-G) shows the organ index of heart, liver, spleen, lung and kidney of mice; (H) shows serum alanine aminotransferase (ALT), aspartate aminotransferase (AST), triglycerides (TG), creatine kinase (CK), urea (UA), lactic dehydrogenase 1 (LDH1) and creatinine (CREA); (I) shows hematoxylin & eosin staining of heart, liver, spleen, lung and kidney; Scale bar: 60 μm.

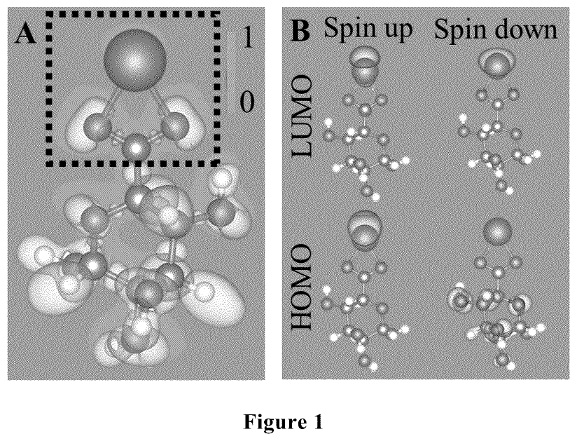

### DETAILED DESCRIPTION OF THE INVENTION

In order to facilitate the understanding of the present invention, the present invention is described below using specific language with reference to certain embodiments. However, it should be understood that these specific embodiments are not intended to limit the scope of the present invention. Any changes and further modifications in the embodiments recited in the present description as well as any further applications of the present invention are generally contemplated by those skilled in the art.

All the test methods in the following examples, unless otherwise specified, are conventional methods. All the reagents and biological materials, unless otherwise specified, can be obtained from commercial sources.

In the following examples, the percentages are all mass percentages unless otherwise specified.

The following examples further illustrate and describe the specific embodiments of the present invention, but the present invention is not limited to the following examples.

The paramagnetic polymer of the present invention is prepared by mixing the sp²-hybridized matrix and Ca cation solution by using mechanical stirring method or microfluidic method. The sp²-hybridized matrix comprises carbon-oxygen double bonds (—C═O). In some embodiments, the sp²-hybridized matrix comprises at least one sp²-hybridized group selected from the group consisting of carboxyl group, aldehyde group or acyl group. In some embodiments, the sp²-hybridized matrix is selected from one or more of polysaccharide uronic acid, acyl and ketone. In some embodiments, the sp²-hybridized matrix is selected from one or more of alginate, carboxymethyl chitosan, polyacrylamide, N-isopropyl acrylamide and hyaluronate. In some embodiments, the sp²-hybridized matrix is sodium alginate. The concentration of the sp²-hybridized matrix solution ranges from 0.3 wt ‰ to 1.3 wt ‰, preferably from 0.3 wt ‰ to 0.7 wt ‰. The concentration of the Ca cation solution ranges from 0.3M to 7M, preferably from 0.3M to 6M.

### Example 1

20 mL of sodium alginate (AlgNa) solution, prepared with 0.3 wt ‰ concentration, is mixed with 15 mL of calcium chloride (CaCl₂) solution, prepared with 0.3M concentration. Using the mechanical stirring method, two solutions are stirred thoroughly to obtain the suspension of Alg-Ca paramagnetic hydrogels.

### Example 2

20 mL of AlgNa solution, prepared with 0.3 wt ‰ concentration, is mixed with 15 mL of CaCl₂) solution, prepared with 0.3M concentration. Using the microfluidic method, one of the above two solutions is dropwise added into the other at a constant rate, with continuous shaking. After the dropwise addition is completed, standing for more than 0.5 h, the suspension of Alg-Ca paramagnetic hydrogels can be obtained.

### Example 3

20 mL of AlgNa solution, prepared with 0.3 wt ‰ concentration, is mixed with 15 mL of CaCl₂ solution, prepared with 1.5M concentration. Using the mechanical stirring method mentioned in example 1 or the microfluidic method mentioned in example 2, the suspension of Alg-Ca paramagnetic hydrogels can be obtained.

### Example 4

20 mL of AlgNa solution, prepared with 0.3 wt ‰ concentration, is mixed with 15 mL of CaCl₂) solution, prepared with 6M concentration. Using the mechanical stirring method mentioned in example 1 or the microfluidic method mentioned in example 2, the suspension of Alg-Ca paramagnetic hydrogels can be obtained.

### Example 5

20 mL of AlgNa solution, prepared with 0.3 wt ‰ concentration, is mixed with 15 mL of CaCl₂) solution, prepared with 7M concentration. Using the mechanical stirring method mentioned in example 1 or the microfluidic method mentioned in example 2, the suspension of Alg-Ca paramagnetic hydrogels can be obtained.

### Example 6

20 mL of AlgNa solution, prepared with 0.7 wt ‰ concentration, is mixed with 15 mL of CaCl₂) solution, prepared with 1M concentration. Using the mechanical stirring method mentioned in example 1 or the microfluidic method mentioned in example 2, the suspension of Alg-Ca paramagnetic hydrogels can be obtained.

### Example 7

20 mL of AlgNa solution, prepared with 0.7 wt ‰ concentration, is mixed with 15 mL of CaCl₂) solution, prepared with 4M concentration. Using the mechanical stirring method mentioned in example 1 or the microfluidic method mentioned in example 2, the suspension of Alg-Ca paramagnetic hydrogels can be obtained.

### Example 8

20 mL of AlgNa solution, prepared with 1.3 wt ‰ concentration, is mixed with 15 mL of CaCl₂) solution, prepared with 0.3M concentration. Using the mechanical stirring method mentioned in example 1 or the microfluidic method mentioned in example 2, the suspension of Alg-Ca paramagnetic hydrogels can be obtained.

### Example 9

20 mL of AlgNa solution, prepared with 1.3 wt ‰ concentration, is mixed with 15 mL of CaCl₂) solution, prepared with 6M concentration. Using the mechanical stirring method mentioned in example 1 or the microfluidic method mentioned in example 2, the suspension of Alg-Ca paramagnetic hydrogels can be obtained.

### Example 10 Purification of the Paramagnetic Polymer

The suspension of Alg-Ca paramagnetic hydrogels is prepared according to examples 1-9. Then the suspension is ultrasonically dispersed for 0.5 h, such that Alg-Ca hydrogels are evenly dispersed in the suspension. Then the suspension container is placed next to a cuboid neodymium magnet (64 mm×54 mm×36 mm), which has a maximum surface magnetic field of ~0.5 T. Alg-Ca hydrogels are attracted towards the magnet and accumulate on the wall of the container. These accumulated Alg-Ca hydrogels are extracted by using a micropipette and settled out by centrifuging for 3 min at 3000 rpm. Then, the supernatant is aspirated and Alg-Ca hydrogels are diluted for ~10 times by volume via adding the Millipore water. After repeating the washing procedure for 3 times, the purified paramagnetic Alg-Ca hydrogels can be obtained.

For all the above examples, sodium alginate can be substituted with carboxymethyl chitosan, polyacrylamide, N-isopropyl acrylamide, hyaluronate and other sp²-hybridized matrices, to prepare paramagnetic polymers.

### Experimental Example 1, Mass Magnetic Susceptibility Test

Multiple types of sp²-hybridized matrices with carbon-oxygen double bonds (—C═O), including sodium alginate (AlgNa) solution, carboxymethyl chitosan (CMCS), polyacrylamide (PAM), N-isopropyl acrylamide (NIPAM) and sodium hyaluronate (HANa), were mixed with calcium chloride (CaCl₂)) solution at 298K, respectively. According to the above embodiments, multiple types of polymers were prepared. As control, AlgNa solution, CMCS, PAM, NIPAM and HANa were mixed with water in the same way.

Superconducting quantum interference device (SQUID) MPMS3 magnetometer (Quantum Design) was used for measuring magnetic susceptibilities. The magnetic field (H) was swept between-30,000 Oe and 30,000 Oe, with the interval of 2500 Oe. The mixed liquor was loaded into a liquid sample holder (C130D, Quantum Design), which was confirmed to be leakproof and sealed tightly before the experiment. All contributions from the sample holder, and the background, i.e., water, were subtracted from their weight-scaled voltage signals, followed by computing the direct current magnetic susceptibilities. The obtained voltage signal was fitted with a SquidLab program, resulting in a magnetization (M) versus magnetic field (H) curve. The mass susceptibility (x) is computed as χ=M/H=a/mH, where a is the moment measured by the SQUID magnetometer and m is the analyte mass.

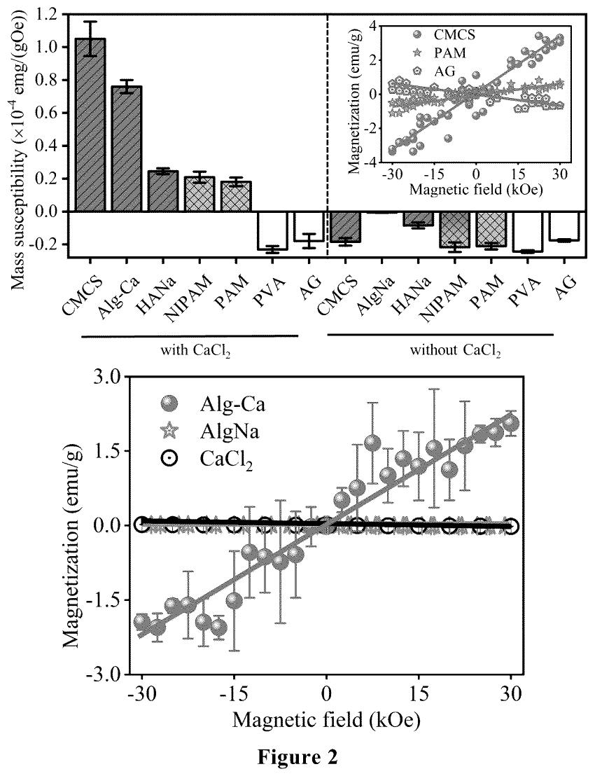

As shown in FIG. 2, the above sp²-hybridized matrices all exhibited strong or very strongly paramagnetism in CaCl₂) solution, as opposed to diamagnetic property in water. The χ of the magnetic Alg-Ca system is (7.6±0.4)×10⁻⁵ emu/(g·Oe), as shown in FIG. 2, which is two orders of magnitude larger than the absolute value of water (−7.2×10⁻⁷ emu/(g·Oe)), showing very strong paramagnetism. By contrast, matrix materials without sp²-hybridized groups, i.e., agarose (AG) and polyvinyl alcohol (PVA), were mixed with CaCl₂) solution and water, respectively in the same way. As shown in FIG. 2, AG and PVA were diamagnetic in both CaCl₂) solution and water.

### Experimental Example 2, T₁ Relaxation Time Test

The suspension of the paramagnetic polymer, i.e., Alg-Ca paramagnetic hydrogel, is prepared according to the above embodiments. The solid content range of the paramagnetic polymer is 0.2-2 mg/ml. The suspension was ultrasonically dispersed in a square plastic container for 30 min. Immediately after that it was placed into a glass NMR tube. T₁ relaxation time was acquired by using a HT-MICNMR-60 system under a magnetic field of 1.5 T at 37° C. Testing parameters were set as follows: repetition time (TR)=10000 ms, echo time (TE)-3 ms. As control, the T₁ relaxation time of ultrapure water was measured under the same condition. Each group was tested more than 3 times, and an average value was taken. As shown in Table 1, the T₁ relaxation time of ultrapure water was 3180±60 ms, and the presence of Alg-Ca hydrogels with different solid contents shortened the T₁ relaxation time to different extents, i.e., 2430±405 ms with 0.2 mg/ml, 1940=450 ms with 1 mg/ml, and 1250±50 ms with 2 mg/ml.

According to Equation 1-1, it is calculated that the Alg-Ca hydrogel of 2 mg/ml shortens the T₁ relaxation time of ultrapure water by 60%.

$$E \% = ( \frac{{T}_{Water} - {T}_{1}}{{T}_{W a t e r}} ) \times \% \qquad 1 - 1$$

Where E % is the efficiency of shortening relaxation time of water, T_{water} and T₁ are the T₁ relaxation times of ultrapure water and contrast agent, respectively.

In addition, in order to eliminate the effect of impurities in the raw materials, sodium alginate (AlgNa) solution with 2 mg/ml was prepared as a control group, the T₁ relaxation time was measured under the same condition. As shown in Table 1, the T₁ relaxation time of 2 mg/ml AlgNa solution was 2800 ms. In order to eliminate the size effect, agarose gels with similar size (~hundred microns in length and tens of microns in width) were prepared and suspended in water at a solid content of 2 mg/ml. As shown in Table 1, the T₁ relaxation time measured under the same condition was 2900 ms. Therefore, the possibility can be ruled out that impurities or size effect induced the shortening of T₁ relaxation time of ultrapure water.

TABLE 1
T₁ relaxation time of ultrapure water, AlgNa solution,
agarose gels and Alg-Ca hydrogels with different
solid contents at 1.5 T magnetic field

| Hydrogels/Control | Solid Content | T₁ relaxation time |
|---|---|---|
| groups | (mg/ml) | (ms) |
| Ultrapure water | / | 3180 ± 60 |
| AlgNa solution | 2 | 2800 |
| Agarose gels | 2 | 2900 |
| Alg-Ca hydrogels | 0.2 | 2430 ± 405 |
| | 1 | 1940 ± 450 |
| | 2 | 1250 ± 50 |

### Experimental Example 3, T₁ Relaxation Time Compared with Commercial Contrast Agents

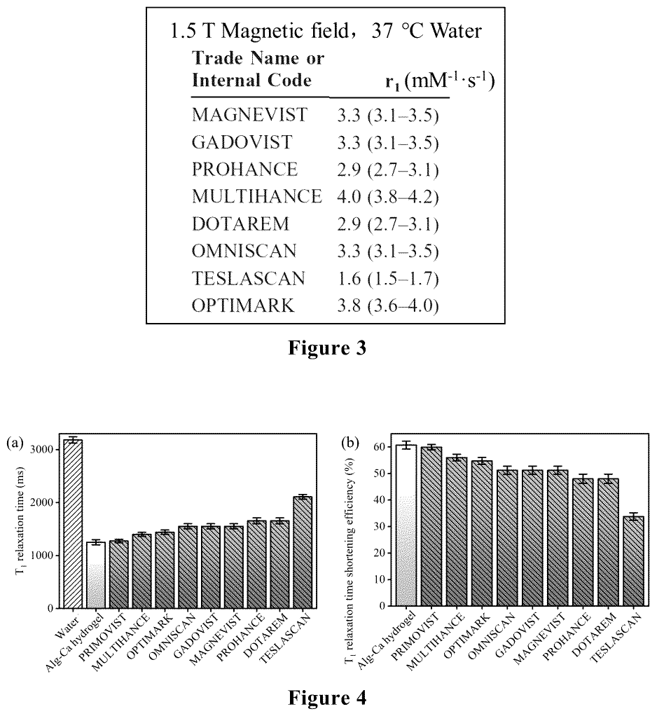

Conventional contrast agents have certain toxicity, so the use of it should be within clinically recommended doses. Generally, the performance of contrast agent is evaluated by the relaxation rate r₁, the ability to shorten the relaxation time per molar concentration. FIG. 3 shows the relaxation rate r₁ of several of the most commonly used commercial contrast agents in a magnetic field of 1.5 T and in water at 37° C. (M. Rohrer et al. 2005). However, since hydrogels contain a large amount of water and cannot be accurately defined per molar concentration, the conventional relaxation rate r₁ is not suitable for evaluating the relaxation effect of paramagnetic hydrogels.

In clinical use, the standard dose for commercial contrast agents, such as: Gd-DTPA, is usually set at 0.1 mmol/kg (L. de Rochefort et al 2008; D. L. Buckley et al. 2008). Thus, the T₁ relaxation time of commercial contrast agents at standard doses can be calculated from Equation 1-2.

$${T}_{1} = 1 / ( \frac{1}{{T}_{W a t e r}} + 0.1 {r}_{1} ) \qquad 1 - 2$$

As shown in FIG. 4 (A), the T₁ relaxation time of 2 mg/ml Alg-Ca hydrogels was comparable to that of commercial contrast agents at standard doses. In addition, the efficiency of shortening relaxation time of water by commercial contrast agents can be calculated from Equation 1-1 and 1-2. As shown in FIG. 4 (B), the efficiency of shortening relaxation time by 2 mg/ml Alg-Ca hydrogels was comparable to that by commercial contrast agents at standard doses.

### Experimental Example 4, T₁ Relaxation Times and MR Images in 114 μT Ultralow Magnetic Field

The suspension of 2 mg/ml Alg-Ca paramagnetic hydrogels was prepared according to the above embodiments and then ultrasonically dispersed for 0.5 h in a square plastic container. T₁ relaxation times and MRI phantom images are tested in self-made ultralow field MRI system at room temperature. The magnetic field intensity was set at 114 μT. In order to compare the MR imaging effect of Alg-Ca hydrogels with that of commercial contrast agents, 0.1 mM and 0.2 mM Gd-DTPA were set as control groups, and T₁ relaxation times and MRI phantom images were measured under the same condition.

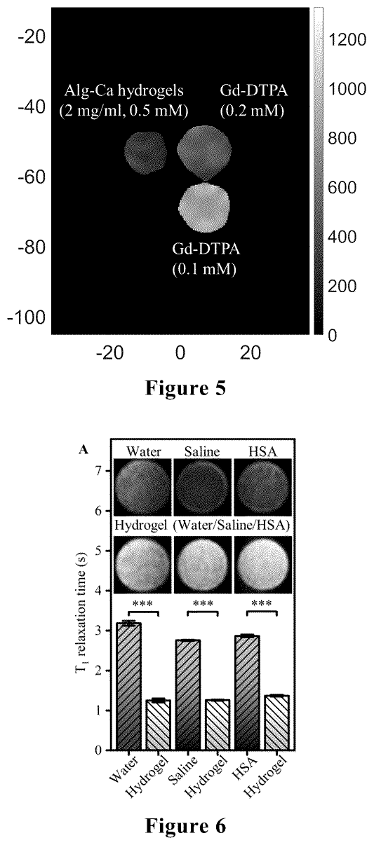

As shown in FIG. 5, the T₁ relaxation time of the 2 mg/ml Alg-Ca hydrogel suspension (296±38 ms) was significantly lower than that of the 0.1 mM Gd-DTPA (844±20 ms) and the 0.2 mM Gd-DTPA (470±12 ms).

### Experimental Example 5, T₁ Relaxation Times of Alg-Ca Paramagnetic Hydrogels in Different Liquids

The purified Alg-Ca paramagnetic hydrogels, prepared according to the above embodiments, were suspended in pure water, saline and human serum albumin (HSA) solution with a solid content of 2 mg/ml. The HSA solution was prepared by dissolving HSA powders into Millipore water by mass ratio of 5%. The water, saline and HSA solution without hydrogels were set as control groups. Then T₁ relaxation times were measured respectively. As shown in FIG. 6, Alg-Ca hydrogels had significantly shortened T₁ relaxation times in various liquid environments, i.e., 1250=50 ms in water, 1258±15 ms in saline, and 1370±20 ms in HSA solution, when comparing with pure water (3180±60 ms), saline (2755±15 ms) and HSA solution (2866-35 ms). Alg-Ca hydrogels also dramatically brightened the MRI phantom images, as shown in FIG. 6.

### Experimental Example 6, MRI In Vivo

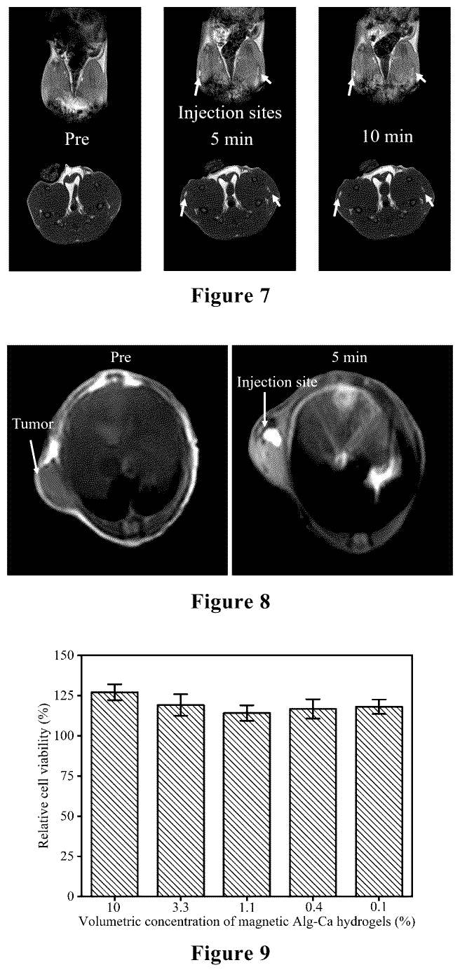

The purified Alg-Ca paramagnetic hydrogels, prepared according to the above embodiments, were diluted by saline to have a solid content of 2 mg/ml. Under Zoletil anesthesia, the living Sprague-Dawley rat (250 g) was fixed onto the supporting substrate. The Alg-Ca hydrogel saline was intramuscularly injected into both left and right back limbs, with a dose of 0.8 μl/g. In vivo MRI was performed by using a 3.0 T MR scanner (Philips Ingenia Elition™). After administrating the Alg-Ca hydrogel, the injection regions exhibited a considerably enhanced contrast, which remained stable for at least 10 min, as shown in FIG. 7.

In another experiment, the Alg-Ca hydrogel/saline was injected to the a Huh7 tumor of a Sprague-Dawley rat under Zoletil anesthesia, with a dose of 0.4 μl/g. The MR images were acquired before and post-injection of 5 min. As shown in FIG. 8, the tumor region had a significantly enhanced contrast compared to normal tissues, which could satisfy the need of clinical diagnosis.

### Experimental Example 7, Cytotoxicity Test of Alg-Ca Paramagnetic Hydrogels

NIH-3T3 cells (ATCC) were selected as representative cells for measuring the cytotoxicity of Alg-Ca paramagnetic hydrogels prepared. NIH-3T3 cells were cultured in the DMEM medium, with 10% fetal bovine serum (FBS) and 1% penicillin and streptomycin. NIH-3T3 cells were initially cultured in a humidified incubator with 5% CO₂ at 37° C. The NIH-3T3 cells were seeded in a 96 well plate with a cell density of ~5×10⁴/ml for 24 h. After that, the cell culture medium, with different concentrations of paramagnetic Alg-Ca hydrogel suspension, was respectively added into the 96 well plate for incubation for another 24 h. The hydrogel concentration (%) was set as: 10, 3.3, 1.1, 0.4, 0.1. NIH-3T3 cells cultured with the pure medium were set as control groups for the cytotoxicity experiments. Then, cell viability of NIH-3T3 cells was investigated by using the Cell Counting Kit-8 (CCK-8) assay. These cells were incubated in the cell culture medium containing 10% of CCK-8 for 4 h, and the absorbance at the wavelength of 450 nm (I₄₅₀) was measured by using a Synergy H1 multi-detection microplate reader. The cell survival rate R_{c} was calculated via dividing the I₄₅₀ of the experimental group by the I₄₅₀ of the control group. Cellular experiments showed that the Alg-Ca hydrogel did not decrease the NIH-3T₃ cell viability even at a high volumetric concentration of ~10%, as shown in FIG. 9, demonstrating that Alg-Ca hydrogel of the present invention was highly biocompatible.

### Experimental Example 8, In Vivo Biocompatibility Study of Alg-Ca Paramagnetic Hydrogels

Eight-week-old male C57B6/J mice were purchased from GemPharmatech Co. Ltd (China). The Alg-Ca paramagnetic hydrogel prepared was intravenously injected into tails of mice. All mice were randomly divided into 4 groups, and were injected with different hydrogel concentration at 0 μL/g, 0.08 μL/g, 0.4 μL/g or 0.8 μL/g. The mice were normally fed for 7 days with their body weight monitored. At the end of the experiment, all mice were sacrificed to collect the organs and tissues, including heart, liver, spleen, lung, kidneys and muscle for histological examination. In the meantime, their blood was collected in 1.5 mL tube, treated with 5% EDTA-2K anticoagulant for complete blood count. The blood samples were centrifuged at 4° C., 3500×g for 15 min to collect serum for biochemical analysis. All biochemical parameters were measured by an automated biochemical analyzer (HITACHI 7020, Japan). All organs and tissues were fixed in 4% paraformaldehyde for 24 h and embedded in paraffin. Then, 5 μm-paraffin tissue sections were dewaxed, rehydrated and stained with hematoxylin & eosin. All sections were examined under the microscope.

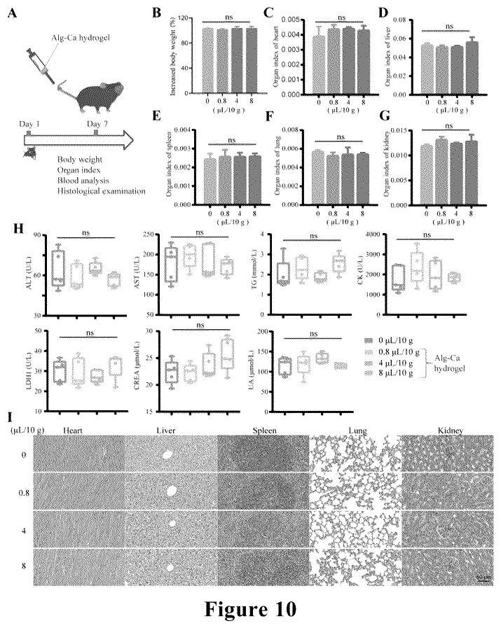

The results of in vivo biocompatibility experiments are shown in FIG. 10. Alg-Ca paramagnetic hydrogels had no noticeable toxicity even at the highest dose. There were no changes in the histology of the blood or tissues. Animal experiments confirmed that the Alg-Ca paramagnetic hydrogel of the present invention was highly biocompatible.

The examples of the present description are only for elaboration of the content of the present invention, instead of imposing any limitation on the present invention. Therefore, any change equivalent to the meanings of the claims of the present invention and within the scope thereof should all be considered as being included in the scope of the claims of the present invention.

All non-patent documents and patent documents cited in the present description are incorporated herein by reference as if each individual non-patent document or patent document is specifically and individually indicated to be incorporated herein by reference. In addition, any theory, mechanism, proof or discovery described herein is intended to further enhance the understanding of the present invention, but is not intended to limit the present invention to such theory, mechanism, proof or discovery in any way. Although the present invention has been illustrated and described in detail in the drawings and the description, the drawings and the description should be considered illustrative rather than restrictive.

### REFERENCES

J. Wahsner, et al., Chemistry of MRI contrast agents: Current challenges and new frontiers. Chemical Reviews 119, 957-1057, 2019
V. M. Runge, Critical questions regarding gadolinium deposition in the brain and body after injections of the gadolinium-based contrast agents, safety, and clinical recommendations in consideration of the EMA's pharmacovigilance and risk assessment committee recommendation for suspension of the marketing authorizations for 4 linear agents. Investigative radiology 52, 2017
M. Rohrer, H. Bauer, J. Mintorovitch, M. Requardt, H. J. Weinmann, Comparison of magnetic properties of MRI contrast media solutions at different magnetic field strengths. Investigative radiology 40, 715-724, 2005
L. de Rochefort et al., In vivo quantification of contrast agent concentration using the induced magnetic field for time-resolved arterial input function measurement with MRI. Medical physics 35, 5328-5339, 2008
D. L. Buckley, L. E. Kershaw, G. J. Stanisz, Cellular-interstitial water exchange and its effect on the determination of contrast agent concentration in vivo: dynamic contrast-enhanced MRI of human internal obturator muscle. Magnetic resonance in medicine 60, 1011-1019, 2008

1. An MRI contrast agent, comprising:
a paramagnetic polymer prepared by mixing an sp2-hybridized matrix and a salt solution of Ca cations.

2. (canceled)

3. The MRI contrast agent of claim 1, wherein
the sp2-hybridized matrix comprises carbon-oxygen double bonds (—C═O).

4. The MRI contrast agent of claim 3, wherein
the sp2-hybridized matrix comprises at least one sp2-hybridized group selected from the group consisting of carboxyl group, aldehyde group or acyl group.

5. (canceled)

6. The MRI contrast agent of claim 5, wherein
the sp2-hybridized matrix is selected from one or more of alginate, carboxymethyl chitosan, polyacrylamide, N-isopropyl acrylamide and hyaluronate.

7. (canceled)

8. The MRI contrast agent of claim 1, wherein
the solution concentration of the sp2-hybridized matrix ranges from 0.3 wt ‰ to 1.3 wt ‰.

9. The MRI contrast agent of claim 8, wherein
the solution concentration of the sp²-hybridized matrix ranges from 0.3 wt ‰ to 0.7 wt ‰.

10. The MRI contrast agent of claim 1, wherein
the concentration of the Ca cation solution ranges from 0.3M to 7M.

11. The MRI contrast agent of claim 10, wherein
the concentration of the Ca cation solution ranges from 0.3M to 6M.

12. The MRI contrast agent of claim 1, wherein
a microfluidic method is used for preparation wherein one of the sp²-hybridized matrix and the Ca cation solution is dropped into the other at a constant rate.

13. The MRI contrast agent of claim 1, wherein
a mechanical stirring method is used for preparation.

14. An MRI contrast agent, comprising:
a paramagnetic polymer prepared by mixing sodium alginate solution and a salt solution of Ca cations.

15. The MRI contrast agent of claim 14, wherein
the solution concentration of the sodium alginate solution ranges from 0.3 wt ‰ to 1.3 wt ‰.

16. The MRI contrast agent of claim 15, wherein
the solution concentration of the sodium alginate solution ranges from 0.3 wt ‰ to 0.7 wt ‰.

17. The MRI contrast agent of claim 16, wherein
the concentration of the Ca cation solution ranges from 0.3M to 7M.

18. The MRI contrast agent of claim 16, wherein
the concentration of the Ca cation solution ranges from 0.3M to 6M.

19. The MRI contrast agent of claim 14, wherein
a microfluidic method is used for preparation wherein one of the sodium alginate solution and the Ca cation solution is dropped into the other at a constant rate.

20. The MRI contrast agent of claim 14, wherein
a mechanical stirring method is used for preparation.

21. An MRI contrast agent, comprising:
a paramagnetic polymer prepared by mixing an sp²-hybridized matrix and a salt solution of Ca cations, and the solid content of the paramagnetic polymer ranges between 0.2-2 mg/ml.

22. The MRI contrast agent of claim 21, wherein
the sp²-hybridized matrix comprises carbon-oxygen double bonds (—C═O).

23. The MRI contrast agent of claim 21, wherein the sp²-hybridized matrix is sodium alginate.
